(19) United States
(12) Patent Application Publication
(10) Pub. No.: US 2022/0183958 A1
(43) Pub. Date: Jun. 16, 2022

(54) Rhamnolipids as deposition aid

(71) Applicant: Evonik Operations GmbH, Essen (DE)

(72) Inventors: Jochen KLEINEN, Heinsberg (DE); Kathrin Daniela Brandt, Duesseldorf (DE); Meike Schulz, Essen (DE); Alina Muss, Essen (DE)

(73) Assignee: Evonik Operations GmbH, Essen (DE)

(21) Appl. No.: 17/436,104

(22) PCT Filed: Feb. 21, 2020
(86) PCT No.: PCT/EP2020/054623

§ 371 Date: Sep. 3, 2021

(30) Foreign Application Priority Data
Mar. 7, 2019 (EP) 19161191.2

(51) Int. Cl. A61K 8/90; A61Q 5/12

(52) U.S. Cl. CPC A61K 8/90; A61Q 5/12

(57) ABSTRACT

Rhamnolipids are used for the deposition of at least one substance from a medium onto a surface.

### FIELD OF THE INVENTION

The invention relates to the use of rhamnolipids for the deposition of at least one substance from a medium onto a surface.

### PRIOR ART

The object of the invention was to identify substances which promote the deposition of at least one substance from a medium onto a surface, in particular skin and hair.

In many applications, substances are deposited from a formulation onto a surface in order to impart a desired property to the surface.

The deposition of conditioning agents, for instance polyguats, on skin and hair, leads for example to improved sensory effects, which is why shampoo formulations often contain such conditioning agents.

In the vast majority of applications, however, the theoretically available total amounts of substance to be deposited are not deposited. A maximization of the deposition of substance is desirable in order to be able to work in a resource-conserving manner.

### DESCRIPTION OF THE INVENTION

Surprisingly, it has been found that an excellent deposition is achieved when using rhamnolipids.

The present invention therefore relates to the use of rhamnolipids for the deposition of at least one substance from a medium onto a surface.

An advantage of the invention is that a very good sensory effect is achieved with the use according to the invention.

A further advantage of the invention is that properties such as combability, softness, shapeability, shine, manageability and disentanglability of hair can be improved with the use according to the invention.

Yet another advantage of the invention is that the irritant effect on the skin and mucous membranes is low with the use according to the invention.

A further advantage of the invention is that compositions based entirely on renewable raw materials can be used in the use according to the invention.

Yet another advantage of the present invention is that it is possible to provide formulations that are free of silicone but nonetheless have good conditioning properties on skin and hair.

Yet another advantage of the invention is that a large foam volume can be achieved with the use according to the invention.

A further advantage is that the foam volume of the inventive formulation is not adversely affected, even in the presence of sebum on skin and hair.

Yet another advantage of the invention is that a particularly good foam creaminess can be achieved with the use according to the invention.

A further advantage of the invention is that formulations which are readily thickened are produced with the use according to the invention.

As an additional advantage, the outstanding compatibility of the rhamnolipid can be mentioned. A further advantage of the present invention is that it reduces the combing forces on wet and dry hair.

The use according to the invention is described hereinafter by way of example, without any intention of limiting the invention to these illustrative embodiments. Where ranges, general formulae or classes of compounds are specified below, these are intended to encompass not only the corresponding ranges or groups of compounds which are explicitly mentioned but also all subranges and subgroups of compounds which can be obtained by removing individual values (ranges) or compounds. Where documents are cited within the context of the present description, the entire content thereof is intended to be part of the disclosure of the present invention. Where, in the context of the present invention, compounds, for instance organomodified polysiloxanes, are described which can have different units multiple times, then these can occur in random distribution (random oligomer) or ordered (block oligomer) in these compounds. The information regarding the number of units in such compounds is to be understood as an average averaged over all corresponding compounds.

The use according to the invention is, if the surface is that of a living body, a non-therapeutic use. In particular, in this context, the use according to the invention is a cosmetic use.

Unless stated otherwise, all percentages (%) given are percentages by mass.

The present invention relates to the use of rhamnolipids for the deposition of at least one substance from a medium onto a surface.

The term “rhamnolipid” in the context of the present invention encompasses rhamnolipids, protonated forms thereof and also in particular salts thereof.

The term “rhamnolipid” in the context of the present invention is understood to mean particularly mixtures of compounds of the general formula (I) and salts thereof,

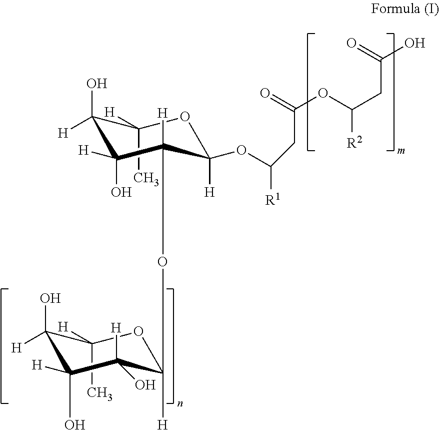

wherein

m=2, 1 or 0,

n=1 or 0,

R¹ and R²=mutually independently identical or different organic radical having 2 to 24, preferably 5 to 13 carbon atoms, in particular optionally branched, optionally substituted, particularly hydroxy-substituted, optionally unsaturated, in particular optionally mono-, bi- or tri-unsaturated alkyl radical, preferably selected from the group consisting of pentenyl, heptenyl, nonenyl, undecenyl and tridecenyl and (CH₂)ₒ—CH₃ where o=1 to 23, preferably 4 to 12.

If n=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where n=1.

The term “mono-rhamnolipid” in the context of the present invention is understood to mean compounds of the general formula (I) or salts thereof, where n=0.

Distinct rhamnolipids are abbreviated according to the following nomenclature:

“diRL-CXCY” is understood to mean di-rhamnolipids of the general formula (I), in which one of the radicals R¹ and R²=(CH₂)ₒ—CH₃ where o=X-4 and the remaining radical R¹ or R²=(CH₂)ₒ—CH₃ where o=Y-4.

“monoRL-CXCY” is understood to mean mono-rhamnolipids of the general formula (I), in which one of the radicals R¹ and R²=(CH₂)ₒ—CH₃ where o=X-4 and the remaining radical R¹ or R²=(CH₂)ₒ—CH₃ where o=Y-4.

The nomenclature used therefore does not distinguish between “CXCY” and “CYCX”, For rhamnolipids where m=0, monoRL-CX or diRL-CX is used accordingly.

If one of the abovementioned indices X and/or Y is provided with “:Z”, this signifies that the respective radical R¹ and/or R² is equal to an unbranched, unsubstituted hydrocarbon radical having X-3 or Y-3 carbon atoms having Z double bonds.

To determine the content of rhamnolipids in the context of the present invention, only the mass of the rhamnolipid anion is considered, i.e. “general formula (I) less one hydrogen”.

To determine the content of rhamnolipids in the context of the present invention, all rhamnolipids are converted by acidification into the protonated form (cf. general formula (I)) and quantified by HPLC.

It is preferred according to the invention that the rhamnolipids used contain 51% by weight to 95% by weight, preferably 70% by weight to 90% by weight, particularly preferably 75% by weight to 85% by weight, of diRL-C10C10, wherein the percentages by weight refer to the sum total of all rhamnolipids used.

It is preferred according to the invention that the rhamnolipids used contain 0.5% by weight to 9% by weight, preferably 0.5% by weight to 3% by weight, particularly preferably 0.5% by weight to 2% by weight, of monoRL-C10C10, wherein the percentages by weight refer to the sum total of all rhamnolipids used.

A preferred use according to the invention is characterized in that the weight ratio of all di-rhamnolipids used to all mono-rhamnolipids used is greater than 51:49, in particular greater than 91:9, preferably greater than 97:3, particularly preferably greater than 98:2.

It is preferred according to the invention that the rhamnolipids used contain 0.5 to 25% by weight, preferably 5% by weight to 15% by weight, particularly preferably 7% by weight to 12% by weight, of diRL-C10C12, wherein the percentages by weight refer to the sum total of all rhamnolipids used. It is preferred according to the invention that the rhamnolipids used comprise 0.1% by weight to 5% by weight, preferably 0.5% by weight to 3% by weight, particularly preferably 0.5% by weight to 2% by weight, of monoRL-C10C12 and/or, preferably and, 0.1% by weight to 5% by weight, preferably 0.5% by weight to 3% by weight, particularly preferably 0.5% by weight to 2% by weight, of monoRL-C10C12:1 , where the percentages by weight refer to the sum total of all rhamnolipids used.

A particularly preferred use according to the invention is characterized in that the rhamnolipids used contain

0.5% by weight to 15% by weight, preferably 3% by weight to 12% by weight, particularly preferably 5% by weight to 10% by weight, of diRL-C10C12:1,

0.5 to 25% by weight, preferably 5% by weight to 15% by weight, particularly preferably 7% by weight to 12% by weight, of diRL-C10C12,

0.1% by weight to 5% by weight, preferably 0.5% by weight to 3% by weight, particularly preferably 0.5% by weight to 2% by weight, of monoRL-C10C12 and

0.1% by weight to 5% by weight, preferably 0.5% by weight to 3% by weight, particularly preferably 0.5% by weight to 2% by weight, of monoRL-C10C12:1,

wherein the percentages by weight refer to the sum total of all rhamnolipids used.

It is moreover preferred if the rhamnolipids used according to the invention contain only small amounts of the formula monoRL-CX or diRL-CX. In particular, the rhamnolipids used according to the invention preferably contain

0% by weight to 5% by weight, preferably 0% by weight to 3% by weight, particularly preferably 0.1% by weight to 1% by weight, of diRLC10, wherein the percentages by weight refer to the sum total of all rhamnolipids used.

A use preferred according to the invention is characterized in that the substance is a polymer.

A use preferred according to the invention is characterized in that the substance is selected from the group comprising, preferably consisting of, siloxanes, carbohydrates, in particular polysaccharides, peptides, proteins, protein hydrolysates, polyallylamines, polyimines, polyacrylates and polymethacrylates,

A use preferred according to the invention is characterized in that the substance has at least one selected from the group of ionic, ionisable and protonatable substituents; in particular, it is preferred that the substance is cationic.

A use preferred according to the invention is characterized in that the substance contains at least one selected from protonatable nitrogen and permanently quaternized nitrogen, in particular in that the substance contains at least one quaternary ammonium group.

A use preferred according to the invention is characterized in that the substance is selected from the group comprising, preferably consisting of, guar quats, siloxane quats and cellulose quats.

A use preferred according to the invention is characterized in that the medium is selected from the group comprising, preferably consisting of, surfactant mixtures, emulsions and microemulsions, in particular O/W emulsions, W/O emulsions, water-in-silicone emulsions, silicone-in water emulsions, O/W microemulsions, W/O microemulsions, water-in-silicone microemulsions, silicone-in-water microemulsions, and dispersions.

A use preferred according to the invention is characterized in that the medium is selected from the group comprising, preferably consisting of, cosmetic formulations, in particular shampoos, conditioners, shower gels, bath soaps, bath oils, gels, mousses and powders.

The cosmetic formulations used in the context of the preferred use according to the invention may contain at least one additional component selected from the group of

emollients,

emulsifiers,

thickeners/viscosity regulators/stabilizers,

UV light protection filters,

antioxidants,

hydrotropes (or polyols),

solids and fillers,

film formers,

pearlescence additives,

deodorant and antiperspirant active ingredients,

insect repellents,

self-tanning agents,

preservatives,

conditioning agents,

dyes,

cosmetic active ingredients,

care additives,

superfatting agents,

solvents.

Substances which can be used as exemplary representatives of the individual groups are known to those skilled in the art and can be found for example in German application DE 102008001788.4. This patent application is hereby incorporated as reference and thus forms part of the disclosure. As regards further optional components and the amounts used of these components, reference is made expressly to the relevant handbooks known to those skilled in the art, for example K. Schrader, “Grundlagen and Rezepturen der Kosmetika [Cosmetics—fundamentals and formulations]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

A use preferred according to the invention is characterized in that the rhamnolipids are present in the medium at a concentration of 0.1% by weight to 30% by weight, preferably of 0.5% by weight to 25% by weight, particularly preferably of 1.0% by weight to 12% by weight, wherein the percentages by weight relate to the total medium.

A use preferred according to the invention is characterized in that the substance is present in the medium at a concentration of 0.05% by weight to 5% by weight, preferably of 0.1% by weight to 3% by weight, particularly preferably of 0.15% by weight to 1.5% by weight, wherein the percentages by weight relate to the total medium.

A use preferred according to the invention is characterized in that the pH of the medium at 25° C. is in a range from 3.5 to 9.0, preferably from 3.8 to 7.0, particularly preferably from 4.4 to 6.6.

The “pH” in connection with the present invention is defined as the value which is measured for the relevant composition at 25° C. after stirring for five minutes using a pH electrode calibrated in accordance with ISO 4319 (1977).

A non-therapeutic use preferred according to the invention is characterized in that the surface is selected from skin and hair, in particular hair.

The examples listed below illustrate the present invention by way of example, without any intention of restricting the invention, the scope of application of which is apparent from the entirety of the description and the claims, to the embodiments specified in the examples.

### EXAMPLES

### Example 1: Deposition of Quaternized Quar on Hair

This example shows that the replacement of various surfactants (SLES and different betaines) by rhamnolipids significantly increases the amount of quaternized guar deposited. As rhamnolipids, use was made hereinbelow of RHEANCE® One (Evonik Industries, 50%, INCI: Glycolipids). The formulation constituents are named in the compositions which follow in the form of the commonly acknowledged INCI nomenclature using the English terms. All concentrations in the application examples 1a-e are given in active content.

The extent of the deposition of the guar was determined on the one hand by measuring the zeta potential (surface potential). For this purpose, the zeta potential is measured on the hair fibre. The relationship between the deposited amount of cationic polymer and the zeta potential is described in J. Jachowicz, M. Berthiaume, and M. Garcia Colloid & Polymer Sci 263:847-858 (1985): a less strongly negative zeta potential corresponds to a higher deposition.

The zeta potential of the hair was measured with a Fiber Potential Analyzer (FPA, emtec, Leipzig, Germany). The zeta potential of the hair tresses was determined before the treatment, since the degree of damage influences the zeta potential and, within a group, only hairs of equal damage/zeta potential were used. Counterintuitively, a more strongly negative zeta potential represents less intensively damaged hair. Consequently, a more strongly positive zeta potential can be achieved by deposition of a cationic polymer on less intensively damaged hair.

Two qualities of hair were able to be identified by the pre-measurements, firstly hair with −31.0 mV and more intensively damaged hair with −25.0 my zeta potential.

For the measurement, the hair tresses (Kehrling) were treated with the corresponding formulations in the manner corresponding to the determination of the sensory properties (panel test, see below). To measure the zeta potential of the hair, a hair tress (4 g) was placed in the measuring chamber of the FPA and tap water was flowed through. 1 g of NaCl was dissolved in 2 litres of water to obtain constant water qualities and conductivity values; this water was used for the measurement. The tresses were immersed in the water (1 g NaCl/2 l) before the measurement, in order to wet the hair. For each sample, 10 measurements each were made in the factory-set auto mode and the mean value was subsequently calculated. All values given are mean values; the fluctuation of the individual values was a maximum of 0.5 mV upwards and 0.5 mV downwards; the reproducibility can also be stated as 0.5 mV.

The formulations each contained 0.5% guar and 12% surfactant. The surfactants used were Sodium Laureth Sulfate (SLES), Cocamidopropyl Betaine (CAPB), Cocobetaine (COB) and Glycolipids (RL).

If positive measured values have been obtained, these are listed before the numerical value in order to achieve a better distinguishability of the measured values.

### Example 1 a: Binary Mixture of Sodium Lauryl Ether Sulfate (SLES) and Rhamnolipid (RL) (Values According to Active Content)

| | | | Zeta potential | |
|---|---|---|---|---|
| SLES/% | RL/% | Guar/% | of hair/mV | Remarks |
| — | — | — | −25.0 | Hair untreated |
| 12 | — | 0.5 | −23.3 | |
| 9 | 3 | 0.5 | −11.2 | |
| 6 | 6 | 0.5 | −3.6 | |
| | 12 | 0.5 | +2.6 | |
| — | — | 0.5 | +3.0 | Only guar, maximum |
| | | | | achievable value |

### Example 1 b: Binary Mixture 9% SLES or 9% RL; 3% Cocamidopropyl Betaine (CAPB) or 3% Cocobetaine (COB) (Values According to Active Content)

| | | | Zeta potential | |
|---|---|---|---|---|
| 9% | 3% | Guar/% | of hair/mV | Remarks |
| — | — | — | −31.0 | Hair untreated |
| SLES | CAPB | 0.5 | −6.2 | |
| SLES | CAPB | 0.5 | −6.4 | Checking reproducibility |
| RL | CAPB | 0.5 | +6.6 | |
| SLES | COB | 0.5 | −9.9 | |
| — | — | 0.5 | +8.5 | Only guar, maximum |
| | | | | achievable value |

### Example 1c: Binary Mixture 6% SLES or 6% RL; 6% CAPB or 6% COB (Values According to Active Content)

| | | | Zeta potential | |
|---|---|---|---|---|
| 6% | 6% | Guar/% | of hair/mV | Remarks |
| — | — | — | −25.0 | Hair untreated |
| SLES | CAPB | 0.5 | −15.7 | |
| RL | CAPB | 0.5 | −11.8 | |
| SLES | COB | 0.5 | −18.0 | |
| — | — | 0.5 | +3.0 | Only guar, maximum |
| | | | | achievable value |

### Example 1 d: Partial Replacement in SLES COB Formulations (Values According to Active Content)

| SLES/ | COB/ | RL/ | Guar/ | Zeta potential | |
|---|---|---|---|---|---|
| % | % | % | % | of hair/mV | Remarks |
| — | — | — | — | −25.0 | Hair untreated |
| 6 | 6 | — | 0.5 | −18.0 | |
| 6 | 3 | 3 | 0.5 | −7.0 | |
| 9 | 3 | — | 0.5 | −9.9 | |
| 9 | 1.5 | 1.5 | 0.5 | −7.9 | |
| — | — | — | 0.5 | +3.0 | Only guar, maximum |
| | | | | | achievable value |

Example 1 e: Individual surfactants. 12% surfactants (values according to active content)

| 12% | Guar/% | Zeta potential of hair/mV | Remarks |
|---|---|---|---|
| — | — | −25.0 | Hair untreated |
| SLES | 0.5 | −23.3 | |
| RL | 0.5 | +2.6 | |
| CAPB | 0.5 | −8.7 | |
| — | 0.5 | +3.0 | Only guar, maximum |
| | | | achievable value |

The results of the measurements from examples 1 a to 1 e in each case show that the use of rhamnolipid in the formulations results in a less negative zeta potential and thus a higher deposition of the cationic polymer (guar quat).

### Example 1 f:

The extent of guar deposition was also able to be demonstrated by the improved sensory effect (panel test) by applying different formulations to strands of hair and subsequently evaluating them sensorially. The formulation constituents are named in the compositions which follow in the form of the commonly acknowledged INCI nomenclature using the English terms. All concentrations in the application example 1f are given in per cent by weight.

For the evaluation by the sensory panel, three simple cosmetic shampoo formulations were prepared, examples A, B and C according to the invention, containing rhamnolipid in different concentrations (see table 1).

TABLE 1
Shampoo formulations for testing the depositing
properties; values in percent by weight

| Formulation examples | A | B | C |
|---|---|---|---|
| RHEANCE One, 50%, Evonik | 24.0% | 12.0% | 6.0% |
| (INCI: Glycolipids) | | | |
| Texapon ® NSO, 28%, Cognis | — | 21.4% | 32.1% |
| (INCI: Sodium Laureth Sulfate) | | | |
| Jaguar ® C-162, 100%, Solvay | 0.5% | 0.5% | 0.5% |
| (INCI: Hydroxypropyl Guar | | | |
| Hydroxypropyltrimonium Chloride) | | | |

| | |
|---|---|
| Water, demineralized | to 100.0% |
| Citric acid | to pH 6.0 |

### Test Conditions For the Sensory Hair Tress Test:

For the sensory hair tress test, European hair from Kerling (prefabricated hair bundles, 18 cm long, 4 g; predamaged in a standardized manner by bleaching) and also toothed plastic combs (17 cm long) with a fine side (about 9.5 teeth/cm) and a rough side (about 4 teeth/cm) are used.

The hair tresses are treated for the sensory hair tress test as follows with the shampoo formulations described in table 1:

### Standardized Treatment of Predamaged Hair Tresses With Shampoo Formulations:

The hair tresses are wetted under warm, running water. The excess water is pressed out gently by hand, and then the shampoo is applied and worked gently into the hair (0.5 g of formulation per hair tress (2 g)). After a contact time of 1 min, the hair is rinsed for 1 min under running water. Subsequently, the sensory evaluation of the hair tresses is carried out by four test persons under standardized conditions at 50% air humidity and 25° C.

The hair tresses are evaluated by the panel as follows:

### Evaluation of the Hair Tresses by the Sensory Panel:

The test criteria are as follows: disentanglability, wet combability and wet feel of the hair. The sensory evaluations are made using grades awarded on a scale from 1 to 5, with 1 being the worst evaluation and 5 being the best evaluation. The individual test criteria each receive their own evaluation. Each panel participant evaluates all criteria for all test formulations. Each hair tress is only used once here.

Details of the evaluation criteria are described in DE 103 27 871.

The results of the sensory evaluation by the panel of the treatment performed as described above of the hair tresses with the inventive formulations A, B and C are shown in table 2.

TABLE 2
Result of sensory evaluation as mean value from 4 subjects

| | | Wet | Wet |
|---|---|---|---|
| | Disentanglability | combability | feel |
| Formulation A | 3.1 | 3.4 | 3.5 |
| (according to the invention) | | | |
| Formulation B | 1.9 | 2.0 | 2.3 |
| (according to the invention) | | | |
| Formulation C | 1.9 | 1.9 | 1.9 |
| (according to the invention) | | | |

The results of the sensory panel show that increasing the proportion of rhamnolipid in the formulation achieves a better evaluation by the sensory panel for all test criteria.

### Example 2: Deposition of Polyquaternium-10 and Silicone Quat on Hair

This example shows that the use of rhamnolipid instead of Sodium Laureth Sulfate (SLES) significantly increases the amount of deposited quaternized hydroxyethyl cellulose. This was confirmed in the sensory panel test as follows. The formulation constituents are named in the compositions which follow in the form of the commonly acknowledged INCI nomenclature using the English terms. All concentrations in the application example 2 are given in per cent by weight. For the evaluation by the sensory panel, two simple cosmetic shampoo formulations were prepared, the inventive example and the non-inventive example E (see table 3).

TABLE 3
Shampoo formulations for testing the depositing properties.

| Formulation examples | D | E |
|---|---|---|
| RHEANCE One, | 18.0 | — |
| (INCI: Rhamnolipid) | | |
| Texapon ® NSO, 28%, Cognis | — | 32.1% |
| (INCI: Sodium Laureth Sulfate) | | |
| TEGO ® Betaine F 50, 38%, Evonik | 7.9% | 7.9% |
| (INCI: Cocamidopropyl Betaine) | | |
| Polymer JR 400, 100%, Amerchol | 0.2% | 0.2% |
| (INCI: Polyquaternium-10) | | |
| ABIL ® ME 45, 30%, Evonik | 3.3% | 3.3% |
| (INCI: Silicone Quaternium-22; Polyglyceryl-3 | | |
| Caprate; Dipropylene Glycol; Cocamidopropyl | | |
| Betaine) | | |

| | |
|---|---|
| Water, demineralized | to 100.0% |
| Citric acid | to pH 6.0 |

The experimental conditions for the sensory hair tress test described in example 1 f apply. The pre-damaged hair tresses are then, as also described in example 1 f, treated with the shampoo formulations from table 3.

The sensory evaluation is carried out under standardized conditions at 50% air humidity and 25° C. as described in example 1 f.

The test criteria are as follows: disentanglability, wet combability, wet feel. Details of the evaluation can be found in example 1 f and DE 103 27 871.

The results of the sensory evaluation by the panel of the treatment preformed as described above of the hair tresses with the inventive formulation D and the non-inventive comparative formulation E are shown in table 4.

TABLE 4
Result of sensory evaluation as mean value from 4 subjects

| | | Wet | Wet |
|---|---|---|---|
| | Disentanglability | combability | feel |
| Inventive formulation (D) | 4.3 | 4.1 | 4.1 |
| Non-inventive formulation (E) | 2.6 | 2.6 | 2.1 |

The results of the sensory panel show that the use of rharnnolipid in the formulation (inventive formulation D) leads to a significantly better evaluation than the use of Sodium Laureth Sulfate (non-inventive formulation E), which can be attributed to an increased deposition of the cationic polymer (Polyquatemium-10) in the case of the use of rhamnolipid.

The improved deposition of the cationic polymer can be further shown by measurements of combing forces on hair, as illustrated by the following example:

### Experimental Conditions for Combing Force Measurements

Instrument: Diastron MIT 175

Measurement distance: 16.5 cm

Combing rate: 2000 mm/min

Hair tresses used: Length=approx. 20 cm (total length—18 cm free hair); width=1.5 cm; weight (dry)=3 g

Measurement conditions: T=22° C.

The hair tresses are measured with a residual moisture of 60%, determined by weight determination.

For the experiments, European hair bleached for 4 h from Kerling is used (pre-prepared flat tresses, 1.5 cm wide, 3 g).

### Carrying Out the Combing Force Measurements

1. The hair tress is dipped for 20 sec in a buffer solution (Na citrate, pH=6).

2. The hair tress is precombed by hand until no change in combing resistance can be perceived.

3. The hair tress is clamped in the instrument and the first combing force measurement is carried out. The measurement is repeated a total of 10 times. Before each further measurement, the hair tress is moistened by two sprays from a deodorant pump sprayer with the buffer solution. The mean combing effort in % is determined using the MTT175 software.

Firstly, all hair tresses are measured in the untreated state. Subsequently, the hair tresses are treated with the test formulations as follows:

### Treatment of the Hair Tresses for the Combing Force Measurements

0.5 g of the respective test formulation is used per hair tress (2 g hair/0.5 g solution). The formulation is massaged into the hair for 30 seconds, then allowed to rest for 1 minute and subsequently rinsed for 30 seconds under running tap water at 37° C. The hair tress is then subsequently shampooed a further time for 30 seconds, allowed to rest for 1 minute and filially rinsed for 1 minute under running tap water at 37° C.

### Carrying Out the Combing Force Measurements

Points 1-3 are repeated for carrying out the combing force measurement with the test formulations. 4 hair tresses are measured for each test formulation. The mean combing effort in % is subsequently determined using the MIT175 software.

Using the described method, the combing forces for the formulation A containing rhamnolipid (according to the invention) were compared with the comparative formulation B (not according to the invention) which contains Sodium Laureth Sulfate instead of rhamnolipid. The results are shown in table 5.

TABLE 5
Result of the combing force measurements as mean value of 4 individual
values; the reduction in combing effort is shown in %

| | Reduction in combing effort [%] |
|---|---|
| Inventive formulation (D) | 49 (±7) |
| Non-inventive formulation (E) | 18 (±12) |

The results show that the formulation A according to the invention has a more pronounced reduction in the combing forces than the comparative formulation B.

### Further Formulation Examples

The formulation constituents are named in the compositions which fallow in the form of the commonly acknowledged INCI nomenclature using the English terms. All concentrations in the application examples are given in active content.

| Example 3 | a | b | c | d | e | f | g | h | i |
|---|---|---|---|---|---|---|---|---|---|
| RHEANCE ® One (Glycolipids) | 2.5 | 2.5 | 2.5 | 2.5 | 2.5 | 2.5 | 2.5 | 2.5 | 2.5 |
| Isostearamide MIPA; Glyceryl Laurate | 1 | 1 | 1 | 1 | 1 | 1 | 1 | 1 | 1 |
| Sorbitan Sesquicaprylate | 0.5 | 0.5 | 0.5 | 0.5 | 0.5 | 0.5 | 0.5 | 0.5 | 0.5 |
| Cocamidopropyl Betaine | 5 | 5 | 5 | 5 | 5 | 5 | 5 | 5 | 5 |
| Disodium Lauryl Sulfosuccinate | 1.5 | 1.5 | 1.5 | 1.5 | 1.5 | 1.5 | 1.5 | 1.5 | 1.5 |
| Sodium Cocoamphoacetate | 3 | 3 | 3 | 3 | 3 | 3 | 3 | 3 | 3 |
| Chitosan | 0.25 | | | | | | | | |
| Hydrolyzed Keratin | | 0.15 | | | | 0.1 | | | |
| Hydrolyzed Wheat Protein | | | 0.32 | | | | | | |
| Hydroxypropyl Guar | | | | 0.4 | | | | 0.1 | |
| Hydroxypropyltrimonium Chloride | | | | | | | | | |
| Polyquaternium-11 | | | | | 0.15 | | | | |
| Polyquaternium-22 | | | | | | 0.4 | | | 0.28 |
| Polyquaternium-6 | 0.25 | | | | | | 0.4 | | |
| Polyquaternium-10 | | | | | | | | 0.3 | |
| Polyquaternium-28 | | | | | 0.15 | | | | 0.22 |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | | | | 0.1 | | | | |
| Polyquaternium-70 (and) Dipropylene Glycol | | | | | | | 0.1 | | |
| Quaternium-80 | | 0.35 | | | | | | | |
| Silicone Quaternium-22 | | | 0.18 | | | | | 0.1 | |
| Methoxy PEG/PPG-7/3 Aminopropyl | | | | 0.1 | | | | | |
| Dimethicone | | | | | | | | | |
| Polymer 50 M | | | | | | | | | |
| Polymer H 75 M | | | | | | | | | |
| Polymer quadrega | | | | | | | | | |

| Preservation and fragrance | q.s. | | | | | | | | |
|---|---|---|---|---|---|---|---|---|---|
| Water | to 100.0% | | | | | | | | |
| Example 3 cont. | j | k | l | m | n | o | p | q | r |
| RHEANCE ® One (Glycolipids) | 2.5 | 2.5 | 2.5 | 2.5 | 2.5 | 2.5 | 2.5 | 2.5 | 2.5 |
| Isostearamide MIPA; Glyceryl Laurate | 1 | 1 | 1 | 1 | 1 | 1 | 1 | 1 | 1 |
| Sorbitan Sesquicaprylate | 0.5 | 0.5 | 0.5 | 0.5 | 0.5 | 0.5 | 0.5 | 0.5 | 0.5 |
| Cocamidopropyl Betaine | 5 | 5 | 5 | 5 | 5 | 5 | 5 | 5 | 5 |
| Disodium Lauryl Sulfosuccinate | 1.5 | 1.5 | 1.5 | 1.5 | 1.5 | 1.5 | 1.5 | 1.5 | 1.5 |
| Sodium Cocoamphoacetate | 3 | 3 | 3 | 3 | 3 | 3 | 3 | 3 | 3 |
| Chitosan | | | | | | | | | |
| Hydrolyzed Keratin | | 0.12 | | | | | | | |
| Hydrolyzed Wheat Protein | | | | 0.25 | | | | | |
| Hydroxypropyl Guar | | | | | | | | | |
| Hydroxypropyltrimonium Chloride | | | | | | | | | |
| Polyquaternium-11 | | | | | | | | | 0.2 |
| Polyquaternium-22 | | | | | 0.15 | | | | |
| Polyquaternium-6 | | | | | | | | | |
| Polyquaternium-10 | | | | | | | 0.2 | | |
| Polyquaternium-28 | | | | | | | | | |
| Polyquaternium-37 (and) PPG-1 Trideceth-6; | 0.19 | | | | | | | | |
| (E) - (and) Glycol Dicaprylate/Dicaprate | | | | | | | | | |
| Polyquaternium-7 | | 0.28 | | | | | | | |
| Polyquaternium-70 (and) Dipropylene Glycol | | | 0.4 | | | | | | 0.2 |
| Quaternium-80 | 0.31 | | | 0.25 | | | | | |
| Silicone Quaternium-22 | | | | | 0.35 | | | | |
| Methoxy PEG/PPG-7/3 Aminopropyl | | | 0.1 | | | 0.5 | | | |
| Dimethicone | | | | | | | | | |
| Polymer 50 M | | | | | | | 0.3 | 0.2 | |
| Polymer H 75 M | | | | | | | | 0.2 | |
| Polymer quadrega | | | | | | | | 0.1 | 0.1 |

| | |
|---|---|
| Preservation and fragrance | q.s. |
| Water | to 100.0% |

| Example 4 | a | b | c | d | e | f | g | h | i |
|---|---|---|---|---|---|---|---|---|---|
| RHEANCE ® One (Glycolipids) | 3.00 | 3.00 | 3.00 | 3.00 | 3.00 | 3.00 | 3.00 | 3.00 | 3.00 |
| TEGO ® Betaine F 50 (Cocamidopropyl Betaine) | 3.00 | 3.00 | 3.00 | 3.00 | 3.00 | 3.00 | 3.00 | 3.00 | 3.00 |
| Helianthus Annuus (Sunflower) Oil | 50.00 | 50.00 | 50.00 | 50.00 | 50.00 | 50.00 | 50.00 | 50.00 | 50.00 |
| ANTIL ® Soft SC (Sorbitan Sesquicaprylate) | 7.00 | 7.00 | 7.00 | 7.00 | 7.00 | 7.00 | 7.00 | 7.00 | 7.00 |
| VARISOFT ® EQ 100 (Bis-(Isostearoyl/Oleyl | 1.00 | 1.00 | 1.00 | 1.00 | 1.00 | 1.00 | 1.00 | 1.00 | 1.00 |
| Isopropyl) Dimonium Methosulfate) | | | | | | | | | |
| Chitosan | 0.20 | | | | | | | | |
| Hydrolyzed Keratin | | 0.30 | | | | | | | |
| Hydrolyzed Wheat Protein | | | 0.30 | | | | | | |
| Hydroxypropyl Guar | | | | 0.20 | | | | | |
| Hydroxypropyltrimonium Chloride | | | | | | | | | |
| Polyquaternium-11 | | | | | 0.10 | | | | |
| Polyquaternium-22 | | | | | | 0.2 | | | |
| Polyquaternium-6 | | | | | | | 0.4 | | |
| Polyquaternium-10 | | | | | | | | 0.7 | |
| Polyquaternium-28 | | | | | | | | | 0.25 |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | | | | | | | | |
| Polyquaternium-70 (and) Dipropylene Glycol | | | | | | | | | |
| Quaternium-80 | | | | | | | | | |
| Silicone Quaternium-22 | | | | | | | | | |
| Methoxy PEG/PPG-7/3 Aminopropyl Dimethicone | | | | | | | | | |
| Polymer 50 M | | | | | | | | | |
| Polymer H 75 M | | | | | | | | | |
| Polymer quadrega | | | | | | | | | |
| Preservation and fragrance | q.s. | q.s. | q.s. | q.s. | q.s. | q.s. | q.s. | q.s. | q.s. |
| Water | to | to | to | to | to | to | to | to | to |
| | 100 | 100 | 100 | 100 | 100 | 100 | 100 | 100 | 100 |
| Example 4 cont. | j | k | l | m | n | o | p | q | r |
| RHEANCE ® One (Glycolipids) | 3.00 | 3.00 | 3.00 | 3.00 | 3.00 | 3.00 | 3.00 | 3.00 | 3.00 |
| TEGO ® Betaine F 50 (Cocamidopropyl Betaine) | 3.00 | 3.00 | 3.00 | 3.00 | 3.00 | 3.00 | 3.00 | 3.00 | 3.00 |
| Helianthus Annuus (Sunflower) Oil | 50.00 | 50.00 | 50.00 | 50.00 | 50.00 | 50.00 | 50.00 | 50.00 | 50.00 |
| ANTIL ® Soft SC (Sorbitan Sesquicaprylate) | 7.00 | 7.00 | 7.00 | 7.00 | 7.00 | 7.00 | 7.00 | 7.00 | 7.00 |
| VARISOFT ® EQ 100 (Bis-(Isostearoyl/Oleyl | 1.00 | 1.00 | 1.00 | 1.00 | 1.00 | 1.00 | 1.00 | 1.00 | 1.00 |
| Isopropyl) Dimonium Methosulfate) | | | | | | | | | |
| Chitosan | | | | | | | | | |
| Hydrolyzed Keratin | | | | | | | | | |
| Hydrolyzed Wheat Protein | | | | | | | | | |
| Hydroxypropyl Guar | | | | | | | | | |
| Hydroxypropyltrimonium Chloride | | | | | | | | | |
| Polyquaternium-11 | | | | | | | | | |
| Polyquaternium-22 | | | | | | | | | |
| Polyquaternium-6 | | | | | | | | | |
| Polyquaternium-10 | | | | | | | | | |
| Polyquaternium-28 | | | | | | | | | |
| Polyquaternium-37 (and) PPG-1 Trideceth-6; | 0.3 | | | | | | | | |
| (E) - (and) Glycol Dicaprylate/Dicaprate | | | | | | | | | |
| Polyquaternium-7 | | 0.4 | | | | | | | |
| Polyquaternium-70 (and) Dipropylene Glycol | | | 0.8 | | | | | | |
| Quaternium-80 | | | | 0.5 | | | | | |
| Silicone Quaternium-22 | | | | | 0.5 | | | | |
| Methoxy PEG/PPG-7/3 Aminopropyl Dimethicone | | | | | | 0.3 | | | |
| Polymer 50 M | | | | | | | 0.5 | | |
| Polymer H 75 M | | | | | | | | 0.5 | |
| Polymer quadrega | | | | | | | | | 0.5 |
| Preservation and fragrance | q.s. | q.s. | q.s. | q.s. | q.s. | q.s. | q.s. | q s | q.s. |
| Water | to | to | to | to | to | to | to | to | to |
| | 100 | 100 | 100 | 100 | 100 | 100 | 100 | 100 | 100 |
| Example 5 | a | b | c | d | e | f | g | h | i |
| RHEANCE ® One (Glycolipids) | 3 | 3 | 3 | 3 | 3 | 3 | 3 | 3 | 3 |
| Sodium Laureth Ether Sulfate | 3 | 3 | 3 | 3 | 3 | 3 | 6 | 6 | 6 |
| Cocamidopropyl Betaine | 3 | | | | | | 3 | | |
| Capryl/Capramidopropyl Betaine | | 3 | | | | | | 3 | |
| Coco-Betaine | | | 3 | | | | | | 3 |
| Sodium Cocoamphoacetate | | | | 3 | | | | | |
| Sodium Cocoamphopropionate | | | | | 3 | | | | |
| Disodium Cocoamphodiacetate | | | | | | 3 | | | |
| Chitosan | 0.5 | | | | | | | | |
| Hydrolyzed Keratin | | 0.5 | | | | | | | |
| Hydrolyzed Wheat Protein | | | 0.5 | | | | | | |
| Hydroxypropyl Guar | | | | 0.5 | | | | | |
| Hydroxypropyltrimonium Chloride | | | | | | | | | |
| Polyquaternium-11 | | | | | 0.5 | | | | |
| Polyquaternium-22 | | | | | | 0.5 | | | |
| Polyquaternium-6 | | | | | | | 0.5 | | |
| Polyquaternium-10 | | | | | | | | 0.5 | |
| Polyquaternium-28 | | | | | | | | | 0.5 |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | | | | | | | | |
| Polyquaternium-70 (and) Dipropylene Glycol | | | | | | | | | |
| Quaternium-80 | | | | | | | | | |
| Silicone Quaternium-22 | | | | | | | | | |
| Methoxy PEG/PPG-7/3 Aminopropyl Dimethicone | | | | | | | | | |
| Polymer 50 M | | | | | | | | | |
| Polymer H 75 M | | | | | | | | | |
| Polymer quadrega | | | | | | | | | |
| Preservation and fragrance | q.s. | q.s. | q.s. | q.s. | q.s. | q.s. | q.s. | q.s. | q.s. |
| Water | to | to | to | to | to | to | to | to | to |
| | 100 | 100 | 100 | 100 | 100 | 100 | 100 | 100 | 100 |
| Example 5 cont. | j | k | l | m | n | o | p | q | r |
| RHEANCE ® One (Glycolipids) | 3 | 3 | 3 | 6 | 6 | 6 | 6 | 6 | 6 |
| Sodium Laureth Ether Sulfate | 6 | 6 | 6 | 3 | 3 | 3 | 3 | 3 | 3 |
| Cocamidopropyl Betaine | | | | 3 | | | | | |
| Capryl/Capramidopropyl Betaine | | | | | 3 | | | | |
| Coco-Betaine | | | | | | 3 | | | |
| Sodium Cocoamphoacetate | 3 | | | | | | 3 | | |
| Sodium Cocoamphopropionate | | 3 | | | | | | 3 | |
| Disodium Cocoamphodiacetate | | | 3 | | | | | | 3 |
| Chitosan | | | | | | | | | |
| Hydrolyzed Keratin | | | | | | | | | |
| Hydrolyzed Wheat Protein | | | | | | | | | |
| Hydroxypropyl Guar | | | | | | | | | |
| Hydroxypropyltrimonium Chloride | | | | | | | | | |
| Polyquaternium-11 | | | | | | | | | |
| Polyquaternium-22 | | | | | | | | | |
| Polyquaternium-6 | | | | | | | | | |
| Polyquaternium-10 | | | | | | | | | |
| Polyquaternium-28 | | | | | | | | | |
| Polyquaternium-37 (and) PPG-1 Trideceth-6; | 0.5 | | | | | | | | |
| (E) - (and) Glycol Dicaprylate/Dicaprate | | | | | | | | | |
| Polyquaternium-7 | | 0.5 | | | | | | | |
| Polyquaternium-70 (and) Dipropylene Glycol | | | 0.5 | | | | | | |
| Quaternium-80 | | | | 0.5 | | | | | |
| Silicone Quaternium-22 | | | | | 0.5 | | | | |
| Methoxy PEG/PPG-7/3 Aminopropyl Dimethicone | | | | | | 0.5 | | | |
| Polymer 50 M | | | | | | | 0.5 | | |
| Polymer H 75 M | | | | | | | | 0.5 | |
| Polymer quadrega | | | | | | | | | 0.5 |
| Preservation and fragrance | q.s. | q.s. | q.s. | q.s. | q.s. | q.s. | q.s. | q.s. | q.s. |
| Water | to | to | to | to | to | to | to | to | to |
| | 100 | 100 | 100 | 100 | 100 | 100 | 100 | 100 | 100 |

| Example 6 | a | b | c | d | e | f | g | h | i |
|---|---|---|---|---|---|---|---|---|---|
| RHEANCE ® One (Glycolipids) | 3 | 8.2 | 1.9 | 2.9 | 0.5 | 3 | 6 | 6 | 3 |
| Sodium Laureth Ether Sulfate | 1.5 | | | | 2.5 | | | | 1 |
| Ammonium Laureth Sulfate | | | 0.5 | | 2.1 | 1 | | 1 | |
| Sodium Cocoyl Glutamate | 3 | 1 | | 2.5 | 0.1 | | 1 | | |
| Sodium Cocoyl Glycinate | | | | 2.5 | | 1 | | | 3 |
| Sodium Cocoyl Sarcosinate | | | 3 | | 1 | 1 | | 2 | |
| Sodium Laureth Sulfate | 1 | | | | | | | | |
| Sodium Lauroyl Methyl Isethionate | | | 1 | | 0.5 | | 3 | | 2 |
| Sodium Lauryl Sulfate | | 1 | | 2.5 | | 1 | | | |
| Sodlum/Disodium Cocoyl Glutamate | 1 | | | | | | | 3 | |
| Stearic Acid | 0.2 | | | 0.1 | | | | | |
| Olea Europaea (Olive) Fruit Oil | | | | 0.1 | | | | | |
| Panthenol | | | 0.2 | | 0.1 | | | | |
| Persea Gratissima (Avocado) Oil | | | 0.1 | | | | | | 0.25 |
| Salicylic Acid | 0.2 | | | | | | 0.2 | | |
| Zinc Pyrithione | | 0.2 | | | | | | | |
| Tetrasodium EDTA | | | | 0.1 | | 0.05 | | 0.1 | |
| Octopirox | | 0.2 | | | 0.25 | | | | |
| Acrylates/C10-30 Alkyl Acrylate | | | | | | | | 0.5 | |
| Crosspolymer | | | | | | | | | |
| Glycerol | | 1 | | | 1 | | 1 | | |
| Glycine Soya (Soybean) Oil | 0.1 | | | 0.1 | | | | 0.1 | |
| Glycol Distearate | | | | | | | | | |
| Dimethicone | | | | | | | | | |
| Creatine | | | | | | 0.1 | | | 0.2 |
| Benzophenone-4 | | | | | | | | | |
| Benzyl Alcohol | | | | | | | | | |
| Butyrospermum Parkii Butter Extract | | | | | | | | | |
| Caffeine | | | | | | | | | 0.25 |
| Cellulose | 0.1 | | | | 0.15 | | | | |
| Olea Europaea (Olive) Fruit Oil | | 0.1 | 0.1 | | | | | | |
| Chitosan | 0.2 | | | | | | | | |
| Hydrolyzed Keratin | | 0.2 | | | | | | | |
| Hydrolyzed Wheat Protein | | | 0.2 | | | | | | |
| Hydroxypropyl Guar | | | | 0.2 | | | | | |
| Hydroxypropyltrimonium Chloride | | | | | | | | | |
| Polyquaternium-11 | | | | | 0.2 | | | | |
| Polyquaternium-22 | | | | | | 0.2 | | | |
| Polyquaternium-6 | | | | | | | 0.2 | | |
| Polyquaternium-10 | | | | | | | | 0.2 | |
| Polyquaternium-28 | | | | | | | | | 0.2 |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | | | | | | | | |
| Polyquaternium-70 (and) Dipropylene Glycol | | | | | | | | | |
| Quaternium-80 | | | | | | | | | |
| Silicone Quaternium-22 | | | | | | | | | |
| Methoxy PEG/PPG-7/3 Aminopropyl | | | | | | | | | |
| Dimethicone | | | | | | | | | |
| Polymer 50 M | | | | | | | | | |
| Polymer H 75 M | | | | | | | | | |
| Polymer quadrega | | | | | | | | | |
| Preservation and fragrance | q.s. | q.s. | q.s. | q.s. | q.s. | q.s. | q.s. | q.s. | q.s. |
| Water | to | to | to | to | to | to | to | to | to |
| | 100 | 100 | 100 | 100 | 100 | 100 | 100 | 100 | 100 |
| Example 6 cont. | j | k | l | m | n | o | p | q | r |
| RHEANCE ® One (Glycolipids) | 3.5 | 2 | 1 | 4.5 | 3.9 | 4.6 | 5.5 | 2.5 | 2 |
| Sodium Laureth Ether Sulfate | | | | | 1 | | | | |
| Ammonium Laureth Sulfate | | | | 1 | | | | | 1 |
| Sodium Cocoyl Glutamate | | | 1 | | | 2 | | 1 | |
| Sodium Cocoyl Glycinate | | 1 | | | | | 1 | | |
| Sodium Cocoyl Sarcosinate | 1 | | | | | 1 | | | |
| Sodium Laureth Sulfate | | 5 | | 4 | | | | | 4.5 |
| Sodium Lauroyl Methyl Isethionate | | | | | | 1.8 | | | |
| Sodium Lauryl Sulfate | | | | | | | | 3.5 | |
| Sodium/Disodium Cocoyl Glutamate | | | | 2.5 | | | 2.5 | | |
| Stearic Acid | 0.1 | | 0.4 | | 0.2 | | | 0.1 | 0.2 |
| Olea Europaea (Olive) Fruit Oil | | | | | | 0.3 | | | |
| Panthenol | | 0.3 | | | | | 0.3 | | |
| Persea Gratissima (Avocado) Oil | | | | | | | | 0.2 | |
| Salicylic Acid | | | | | | 0.3 | | 0.1 | |
| Zinc Pyrithione | 0.2 | | | 0.2 | | 0.2 | | | |
| Tetrasodium EDTA | | | | | | | | | 0.1 |
| Octopirox | | | | 0.1 | | | | 0.1 | |
| Acrylates/C10-30 Alkyl Acrylate | | | | | | | | | |
| Crosspolymer | | | | | | | | | |
| Glycerol | 1 | 3 | | 2 | | 5 | | 3 | 1 |
| Glycine Soya (Soybean) Oil | | | 0.1 | | | | | | |
| Glycol Distearate | | | | | | | | | |
| Dimethicone | | | | 0.3 | | | | | |
| Creatine | | | | | | | | | |
| Benzophenone-4 | | | | | | | | | |
| Benzyl Alcohol | | | 0.1 | | | | | | 0.2 |
| Butyrospermum Parkii Butter Extract | | | | | | | | 0.2 | |
| Caffeine | | | | 0.1 | | | | | |
| Cellulose | | | | | | | | | |
| Olea Europaea (Olive) Fruit Oil | | | | | | | | | |
| Chitosan | | | | | | | | | |
| Hydrolyzed Keratin | | | | | | | | | |
| Hydrolyzed Wheat Protein | | | | | | | | | |
| Hydroxypropyl Guar | | | | | | | | | |
| Hydroxypropyltrimonium Chloride | | | | | | | | | |
| Polyquaternium-11 | | | | | | | | | |
| Polyquaternium-22 | | | | | | | | | |
| Polyquaternium-6 | | | | | | | | | |
| Polyquaternium-10 | | | | | | | | | |
| Polyquaternium-28 | | | | | | | | | |
| Polyquaternium-37 (and) PPG-1 Trideceth-6; | 0.2 | | | | | | | | |
| (E) - (and) Glycol Dicaprylate/Dicaprate | | | | | | | | | |
| Polyquaternium-7 | | 0.2 | | | | | | | |
| Polyquaternium-70 (and) Dipropylene Glycol | | | 0.2 | | | | | | |
| Quaternium-80 | | | | 0.2 | | | | | |
| Silicone Quaternium-22 | | | | | 0.2 | | | | |
| Methoxy PEG/PPG-7/3 Aminopropyl | | | | | | 0.2 | | | |
| Dimethicone | | | | | | | | | |
| Polymer 50 M | | | | | | | 0.2 | | |
| Polymer H 75 M | | | | | | | | 0.2 | |
| Polymer quadrega | | | | | | | | | 0.2 |
| Preservation and fragrance | q.s. | q.s. | q.s. | q.s. | q.s. | q.s. | q.s. | q.s | q.s. |
| Water | to | to | to | to | to | to | to | to | to |
| | 100 | 100 | 100 | 100 | 100 | 100 | 100 | 100 | 100 |

| Example 7 | a | b | c | d | e | f | g | h | i |
|---|---|---|---|---|---|---|---|---|---|
| RHEANCE ® One (Glycolipids) | 7.5 | 5.5 | 5 | 2.8 | 3.9 | 2.9 | 4.5 | 6 | 8 |
| Coco-Glucoside | 10 | 2 | 1 | 1 | | 1 | 6 | 1.5 | 1 |
| Lauryl Glucoside | | 5 | 1 | 5 | 5 | 3 | 2.5 | 1.8 | 1 |
| Decyl Glucoside | | 3 | 1 | 2 | 3 | 2 | 2.5 | 3 | 1 |
| Xanthan Gum | | | 0.5 | | | 0.5 | | | |
| Chitosan | | | | | | | | | |
| Hydrolyzed Keratin | | 0.1 | | | | | | | |
| Hydrolyzed Wheat Protein | | | | | | | | | 0.25 |
| Hydroxypropyl Guar | | | | | | | | | |
| Hydroxypropyltrimonium Chloride | | | | | | | | | |
| Polyquaternium-11 | | | | | | | 0.3 | | |
| Polyquaternium-22 | | | | | | | | | |
| Polyquaternium-6 | | | | | 0.2 | | | 0.1 | |
| Polyquaternium-10 | | | | | | | | | |
| Polyquaternium-28 | 0.1 | 0.3 | 0.1 | | | 0.1 | | | |
| Polyquaternium-37 (and) PPG-1 Trideceth-6; | 0.5 | | | | | | | | |
| (E) - (and) Glycol Dicaprylate/Dicaprate | | | | | | | | | |
| Polyquaternium-7 | | | | 1.2 | | | | | |
| Polyquaternium-70 (and) Dipropylene Glycol | | | | | | | | | |
| Quaternium-80 | | 0.1 | | | 0.1 | 0.1 | | | 0.1 |
| Silicone Quaternium-22 | | | | | | 0.1 | | | |
| Methoxy PEG/PPG-7/3 Aminopropyl Dimethicone | | | | | | | | | |
| Polymer 50 M | | | | | | | | 0.3 | |
| Polymer H 75 M | | | | | | | 0.4 | | |
| Polymer quadrega | | | | | | 0.2 | | | |
| pH | 5 | 5 | 4.8 | 5 | 5.9 | 5 | 5.9 | 4.9 | 4.8 |
| Preservation and fragrance | q.s. | q.s. | q.s. | q.s. | q.s. | q.s. | q.s. | q.s. | q.s. |
| Water | to | to | to | to | to | to | to | to | to |
| | 100 | 100 | 100 | 100 | 100 | 100 | 100 | 100 | 100 |
| Example 7 cont. | j | k | l | m | n | o | p | q | r |
| RHEANCE ® One (Glycolipids) | 5 | 2 | 1.1 | 1.1 | 1.7 | 1.5 | 5.9 | 6.9 | 10 |
| Coco-Glucoside | | | 2 | | 3 | | | 2 | 1 |
| Lauryl Glucoside | | 5 | | 2 | | 3 | 2 | | 1 |
| Decyl Glucoside | | | 5 | 5 | 3 | 5 | 2 | 2 | |
| Xanihan Gum | 0.9 | 0.8 | | 0.9 | | | 0.3 | | |
| Chitosan | | 0.29 | | | 0.1 | | | | |
| Hydrolyzed Keratin | | | | | | | | | |
| Hydrolyzed Wheat Protein | | | 0.25 | | | | | | |
| Hydroxypropyl Guar | | | | | | 0.23 | | | |
| Hydroxypropyltrimonium Chloride | | | | | | | | | |
| Polyquaternium-11 | | | | | | | | | |
| Polyquaternium-22 | | | | | | | | 0.29 | |
| Polyquaternium-6 | | | | | | | | | |
| Polyquaternium-10 | | | | | | | 0.45 | | |
| Polyquaternium-28 | | | | | | 0.2 | | | |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | | | | | | | | |
| Polyquaternium-70 (and) Dipropylene Glycol | | | | | | | | | 0.39 |
| Quaternium-80 | | | | 0.2 | | | | | |
| Silicone Quaternium-22 | | | | 0.15 | | | | | |
| Methoxy PEG/PPG-7/3 Aminopropyl Dimethicone | 0.25 | | | | | | | | |
| Polymer 50 M | | | | | | | | | |
| Polymer H 75 M | | | | | 0.3 | | | | |
| Polymer quadrega | | | | | | | | 0.2 | |
| pH | 5.2 | 6.3 | 5.3 | 4.9 | 5 | 6.5 | 4.5 | 5.2 | 4.7 |
| Preservation and fragrance | q.s. | q.s. | q.s. | q.s. | q.s. | q.s. | q.s. | q.s | q.s. |
| Water | to | to | to | to | to | to | to | to | to |
| | 100 | 100 | 100 | 100 | 100 | 100 | 100 | 100 | 100 |

| Example 8 | a | b | c | d | e | f | g | h | i |
|---|---|---|---|---|---|---|---|---|---|
| RHEANCE ® One (Glycolipids) | 8 | 9 | 10 | 5 | 8 | 4 | 3 | 9 | 6 |
| Palmitamidopropyltrimonium Chloride | 0.5 | | | | | 0.5 | 0.3 | | 0.1 |
| Cocamide DEA | 0.5 | | | 0.5 | | | | | |
| Cocamide MEA | | 0.5 | | | 0.5 | | | | |
| Glycol Distearate | | | | | | 0.3 | | | |
| Disodium PEG-4 Cocamido MIPA-Sulfosuccianate | | | | | | 0.5 | | | |
| Dicaprylylether | | | | | | | | | |
| Isostearamide MIPA | | | | | | 0.2 | | | |
| Oleyl Erucate | | | | | | | | | 1 |
| PEG-200 Hydrogenated Glyceryl Palmate; PEG-7 | | | | | | | | 1 | |
| Glyceryl Cocoate | | | | | | | | | |
| PEG-40 Hydrogenated Castor Oil | | | | | 1 | | | | |
| Polyglyceryl-4 Caprate | 3 | | | | | | | | |
| Propylene Glycol; PEG-55 Propylene Glycol Oleate | | | | | | | | | |
| Prunus Amygdalus Dulcis (Sweet Almond) Oil | 2 | | | | 1 | | 0.5 | | |
| Sodium C14-16 Olefin Sulfonate | | 3 | | 5 | | 5 | 10 | 1 | |
| Sodium Chloride | | | 2 | | | | | | |
| Chitosan | | | | | | | | | |
| Hydrolyzed Keratin | | | | | | | | | |
| Hydrolyzed Wheat Protein | | 0.23 | | | | | | | 0.25 |
| Hydroxypropyl Guar | 0.36 | | | | | | | 0.9 | |
| Hydroxypropyltrimonium Chloride | | | | | | | | | |
| Polyquaternium-11 | | | | | | | 0.2 | | |
| Polyquaternium-22 | 0.28 | | | 0.4 | | | | | |
| Polyquaternium-6 | | | | | | | | 0.1 | |
| Polyquaternium-10 | | | | | 0.4 | | | 0.3 | |
| Polyquaternium-28 | 0.22 | | | | | | | | |
| Polyquaternium-37 (and) PPG-1 Trideceth-6; | | | | | | | | | |
| (E) - (and) Glycol Dicaprylate/Dicaprate | | | | | | | | | |
| Polyquaternium-7 | | 0.28 | | | | | | | |
| Polyquaternium-70 (and) Dipropylene Glycol | | | 0.1 | | | | 0.2 | | |
| Quaternium-80 | | | | | | | | | 0.25 |
| Silicone Quaternium-22 | | | | | | 0.2 | | | |
| Methoxy PEG/PPG-7/3 Aminopropyl Dimethicone | | | | | | | | | |
| Polymer 50 M | | | | | 0.3 | 0.2 | | | |
| Polymer H 75 M | | 0.12 | | 0.1 | | | | | |
| Polymer quadrega | | | | | | 0.1 | 0.9 | | |

| Example 8 cont. | j | k | l | m | n | o | p | q | r |
|---|---|---|---|---|---|---|---|---|---|
| RHEANCE ® One (Glycolipids) | 3.5 | 11.5 | 15 | 10 | 11 | 9.5 | 8.5 | 2.5 | 5.5 |
| Palmitamidopropyltrimonium Chloride | | 1.5 | | | | 1 | | | |
| Cocamide DEA | | | | | | | | | |
| Cocamide MEA | | | | | | | | | |
| Glycol Distearate | | | | | | | | | |
| Disodium PEG-4 Cocamido MIPA-Sulfosuccianate | 0.5 | | | | | | | | |
| Dicaprylylether | | | 0.2 | 0.2 | | | | 0.5 | |
| Isostearamide MIPA | | 1 | | | | | | | |
| Oleyl Erucate | | | | | | | | | |
| PEG-200 Hydrogenated Glyceryl Palmate; PEG-7 | | | | | | | | | |
| Glyceryl Cocoate | | | | | | | | | |
| PEG-40 Hydrogenated Castor Oil | | | | | | | | 5 | 3 |
| Polyglyceryl-4 Caprate | | | | 1 | | | | | |
| Propylene Glycol; PEG-55 Propylene Glycol Oleate | | | | | 0.5 | | | | |
| Prunus Amygdalus Dulcis (Sweet Almond) Oil | | | | | | 0.1 | 0.1 | 0.1 | 0.1 |
| Sodium C14-16 Olefin Sulfonate | 10 | | | | | | | 12 | 10 |
| Sodium Chloride | 1 | | 1.5 | 1 | | 0.25 | | | |
| Chitosan | | | | | | | 0.25 | | |
| Hydrolyzed Keratin | | | | | | | 0.25 | | |
| Hydrolyzed Wheat Protein | | 0.32 | | | | | | | |
| Hydroxypropyl Guar | | | | 0.4 | | | | | |
| Hydroxypropyltrimonium Chloride | | | | | | | | | |
| Polyquaternium-11 | | | | | | | | | 0.25 |
| Polyquaternium-22 | | | | | 0.75 | | | | |
| Polyquaternium-6 | | 0.18 | | | 0.35 | | | | |
| Polyquaternium-10 | | | | | | | | | |
| Polyquaternium-28 | | | | | | | | | 0.55 |
| Polyquaternium-37 (and) PPG-1 Trideceth-6; | 0.39 | | | | | | | | |
| (E) - (and) Glycol Dicaprylate/Dicaprate | | | | | | | | | |
| Polyquaternium-7 | | | | | | | | | 0.1 |
| Polyquaternium-70 (and) Dipropylene Glycol | | | 0.4 | | | | 0.3 | | |
| Quaternium-80 | 0.31 | | | | | | | 0.35 | |
| Silicone Quaternium-22 | | | | | 0.5 | | | | |
| Methoxy PEG/PPG-7/3 Aminopropyl Dimethicone | | | 0.55 | 0.5 | | 0.5 | | | |
| Polymer 50 M | | | | | | | | | |
| Polymer H 75 M | | | | | | | | 0.15 | |
| Polymer quadrega | | | | | | | | | |

1. A method comprising:
depositing at least one substance from a medium onto a surface, wherein the medium comprises a rhamnolipid.

2. The method according to claim 1, wherein the at least one substance is a polymer.

3. The method according to claim 1, wherein the at least one substance is selected from the group consisting of a siloxane, a carbohydrate, a peptide, a protein, a protein hydrolysate, a polyallylamine, a polyimine, a polyacrylate, and a polymethacrylate.

4. The method according to claim 1, wherein the at least one substance has at least one substituent selected from the group consisting of an ionic substituent, an ionisable substituent, and a protonatable substituent.

5. The method according to claim 1, wherein the at least one substance is quaternized.

6. The method according to claim 1, wherein the at least one substance is selected from the group of consisting of a guar quat, a siloxane quat, and a cellulose quat.

7. The method according to claim 1, wherein the medium is selected from the group consisting of a surfactant mixture, an emulsion, and a microemulsion.

8. The method according to claim 1, wherein the medium is selected from the group consisting of cosmetic formulations.

9. The method according to claim 1, wherein the rhamnolipid is present in the medium at a concentration of 0.1% by weight to 30% by weight, of the total medium.

10. The method according to claim 1, wherein the at least one substance is present in the medium at a concentration of 0.05% by weight to 5% by weight, of the total medium.

11. The method according to claim 1, wherein a weight ratio of all di-rhamnolipids used to all mono-rhamnolipids used is greater than 51:49.

12. The method according to claim 1, wherein a pH of the medium at 25° C. is in a range from 3.5 to 9.0.

13. The method according to claim 1, wherein the surface is selected from the group consisting of skin and hair.

14. The method according to claim 5, wherein the at least one substance contains at least one selected from the group consisting of protonatable nitrogen and permanently quaternized nitrogen.

15. The method according to claim 7, wherein the medium is selected from the group consisting of an oil in water (O/W) emulsion, a water in oil (W/O) emulsion, a water-in-silicone emulsion, a silicone-in water emulsion, an oil in water (O/W) microemulsion, a water in oil (W/O) microemulsion, a water-in-silicone microemulsion, a silicone-in-water microemulsion, and a dispersion.

16. The method according to claim 9, wherein the rhamnolipid is present in the medium at a concentration of 1.0% by weight to 12% by weight, of the total medium.

17. The method according to claim 10, wherein the at least one substance is present in the medium at a concentration of 0.15% by weight to 1.5% by weight, of the total medium.

18. The method according to claim 11, wherein the weight ratio of all di-rhamnolipids used to all mono-rhamnolipids used is greater than 98:2.

19. The method according to claim 12, wherein the pH of the medium at 25° C. is in a range from 4.4 to 6.6.

20. The method according to claim 13, wherein the surface is hair.
